(19) United States
(12) Patent Application Publication
(10) Pub. No.: US 2022/0347132 A1
(43) Pub. Date: Nov. 3, 2022

(54) SPRAYABLE LIQUID LIDOCAINE SOLUTION

(71) Applicant: CELISTA PHARMACEUTICALS LLC, Edina, MN (US)

(72) Inventor: Yogesh DANDIKER, Edina, MN (US)

(21) Appl. No.: 17/761,507

(22) PCT Filed: Sep. 17, 2020
(86) PCT No.: PCT/US2020/051193

§ 371 Date: Mar. 17, 2022

Related U.S. Application Data
(60) Provisional application No. 62/902,543, filed on Sep. 19, 2019.

(51) Int. Cl. A61K 31/167; A61K 9/08; A61K 47/10; A61P 23/02; A61K 9/00; A61K 47/38; A61K 47/32; A61K 9/70

(52) U.S. Cl. CPC A61K 31/167; A61K 9/08; A61K 47/10; A61P 23/02; A61K 9/0014; A61K 47/38; A61K 47/32; A61K 9/7015

(57) ABSTRACT

The invention relates to a sprayable liquid solution for the local delivery of lidocaine to the epidermis (skin), gingiva, oral mucosa or mucosal lining of the pharynx, and to a method of providing local anesthesia by administering this composition. The solution comprises at least 40% w/w lidocaine, or about 2% to about 50% w/w/ lidocaine.

### BACKGROUND

Postherpetic neuralgia is a painful condition that affects nerves and skin. It is a complication of herpes zoster, often called shingles. Shingles, also known as acute herpes zoster, is a painful, blistering skin rash caused by a reactivation of dormant varicella-zoster in people who have previously had chicken pox. The virus can remain dormant in nerve cells after infection and reactivate years later.

Postherpetic neuralgia (PHN) is a chronic, painful neuropathic condition that persists for 3 months or longer following an outbreak of shingles. (Sampathkumar P, Drage L A, Martin D P. Herpes zoster (shingles) and postherpetic neuralgia. Mayo Clin Proc. 2009; 84:274-80. Hadley G R, Gayle J A, Ripoll J, et al. Post-herpetic neuralgia: a review. Curr Pain Headache Rep. 2016; 20:17.) Postherpetic neuralgia is the most common complication of shingles, and it occurs when nerves are damaged during a shingles outbreak. Approximately 20-30% of people will have shingles during their lifetime. (Jeon Y H. Herpes zoster and postherpetic neuralgia: practical consideration for prevention and treatment. Korean J Pain. 2015; 28:177-84.) The estimated mean incidence of shingles is about 3.4-4.82 per 1000 person years, increasing to more than 11 per 1,000 person years among persons >80 years old (Johnson μW, et al., Herpes zoster epidemiology, management, and disease and economic burden in Europe: a multidisciplinary perspective, Ther Adv Vaccines 2015; 3(4) 109-120).

PHN is associated with persistent and often refractory neuropathic pain (Jeon et al., 2015). Patients may experience multiple types of pain including a constant deep, aching, or burning pain; a paroxysmal, lancinating pain; hyperalgesia (painful stimuli which are more painful than expected); and allodynia (pain associated with typically non-painful stimuli) (Hadley et al., 2016). PHN pain is typically localized, unilateral and chronic, but may be constant, intermittent, spontaneous or evoked.

PHN is likely to interfere with sleep and daily activities (Nalamachu S, Morley-Forster P. Diagnosing and managing postherpetic neuralgia. Drugs Aging. 2012; 29:863-9), is associated with significantly diminished quality of life and can be a significant economic burden for patients and society. (Gater A, Uhart M, McCool R, Préaud E. The humanistic, economic and societal burden of herpes zoster in Europe: a critical review. BMC Public Health. 2015; 15:193; Panatto D, Bragazzi N L, Rizzitelli E, et al. Evaluation of the economic burden of Herpes Zoster (HZ) infection. Hum. Vaccin. Immunother. 2015; 11:245-62.)

Following an episode of chicken pox, the varicella zoster virus remains latent in the spinal dorsal root ganglia and cranial sensory ganglia (Jeon et al., 2015). When cell-mediated immunity against varicella zoster virus decreases, usually as a result of aging, the virus replicates and spreads along the peripheral nerves to the skin, leading to the characteristic painful erythematous rash in the affected dermatome (shingles) (Jeon et al., 2015).

The pathophysiology of PHN is less well understood; however, it is known that shingles affects the central and peripheral nervous systems, which can subsequently lead to the occurrence of PHN. The two main processes responsible for the development of PHN are sensitization and deafferentation (Jeon et al., 2015). Replication of latent varicella zoster in the sensory ganglia leads to inflammatory neural damage, resulting in acute pain and PHN.

During the course of shingles, both the peripheral and central nervous systems can be injured (Jeon et al., 2015). The net effect is spontaneous pain, allodynia, and hyperalgesia (Jeon et al., 2015).

The pharmacological treatment of PHN may include a variety of medications including gabapentin (Beal B, Moeller-Bertram T, Schilling J M, Wallace M S. Gabapentin for once-daily treatment of post-herpetic neuralgia: a review. Clin. Interv. Aging. 2012; 7:249-55) and pregabalin, other anticonvulsants (carbamazepine), tricyclic antidepressants (amitriptyline, nortriptyline, doxepin), tramadol, opioids, topical analgesics such as lidocaine (Katz N P, Gammaitoni A R, Davis M W, et al. Lidocaine patch 5% reduces pain intensity and interference with quality of life in patients with postherpetic neuralgia: an effectiveness trial. Pain Med. 2002; 3:324-32) and capsaicin (Peppin J F, Pappagallo M. Capsaicinoids in the treatment of neuropathic pain: a review. Ther. Adv. Neurol. Disord. 2014; 7:22-32). The adverse event profiles of many oral medications often limits their practical use, and a combination of both topical and systemic agents may be required for maximum benefit (White W T, Patel N, Drass M, Nalamachu S. Lidocaine patch 5% with systemic analgesics such as gabapentin: a rational polypharmacy approach for the treatment of chronic pain. Pain Med. 2003; 4:321-30; Bruckenthal P, Barkin R L. Options for treating postherpetic neuralgia in the medically complicated patient. Ther. Clin. Risk Manag. 2013; 9:329-40; Hadley et al., 2016).

The antiviral medications valacyclovir (Valtrex® (valacyclovir hydrochloride) Caplets, GlaxoSmithKline Research Triangle Park, N.C.) and acyclovir (Zovirax® (acyclovir) Capsules, GlaxoSmithKline Research Triangle Park, N.C.) are indicated for the treatment of shingles in immunocompetent adults (Valtrex® Caplets Prescribing Information, 2013; Zovirax® Capsules Prescribing Information, 2005). Treatment of acute varicella with acyclovir attenuates acute illness but does not prevent herpes zoster. (Douglas M W, Johnson μW, Cunningham A L. Tolerability of treatments for postherpetic neuralgia. Drug Saf. 2004; 27:1217-33.) Treatment of herpes zoster with acyclovir or its derivatives minimizes symptoms and may reduce the rate of PHN. However, new treatment options are needed to provide quick and effective pain relief.

### SUMMARY

The invention provides an improved topical lidocaine formulation comprising at least 40% w/w lidocaine. The invention relates to a sprayable liquid solution for the local delivery of lidocaine to the epidermis (skin), gingiva, oral mucosa or mucosal lining of the pharynx, and to a method of providing local anesthesia by administering this composition. The composition contains little or no water, so dries quickly. The invention also relates to a method of providing local anesthesia or pain relief for allodynia (painful hypersensitivity), for pain associated with post-herpetic neuralgia PHN, a method of providing pain relief or local anesthesia to the gingiva or oral mucosa, a method of providing anesthesia to an area of the gingiva prior to a dental procedure, a method of providing anesthesia to an injection area of the skin prior to an injection, or to a method of providing local anesthesia or preventing pain during an endoscopy or intubation.

### BRIEF DESCRIPTION OF THE DRAWINGS

The foregoing and other features and aspects of the present invention can be better understood from the following description of embodiments and as illustrated in the accompanying drawings. The accompanying drawings, which are incorporated herein and form a part of the specification, further serve to illustrate the principles of the present invention.

FIG. 1 is a graph showing the cumulative amount of lidocaine in the receiver compartment per unit area (μg/cm²) from representative formulations over the 24 h experimental period in the experiment described in Example 4A. Each point represents the mean amount±SEM detected in the receiver fluid across human skin from one skin donor (n=5-6).

FIG. 2 is a graph depicting the amount of lidocaine (m) penetrated into the S. corneum, epidermis and partial dermis following application of formulations outlined in Table 4 and Lidoderm patch over the 24 h experimental period in the experiment described in Example 4B. Each point represents the mean amount±SEM (n=5-6).

### DETAILED DESCRIPTION

The present invention is directed to a sprayable liquid lidocaine solution for topical application to provide local delivery of lidocaine. The concentration of lidocaine in the sprayable liquid solution is at least 40% w/w for most applications, but may be about 2% to about 50% for e.g., formulations for application to the oral mucosa or gingiva. This composition provides an effective dose of lidocaine in a small volume spray that is easy to apply and provides quick delivery of a high concentration of lidocaine and excellent skin penetration to provide fast and effective pain relief. In addition, the composition contains little or no water (less than about 10% w/w, about 5% w/w or 1% w/w), so dries quickly.

The composition can contain a film forming excipient that leaves a washable film on the surface of the skin to protect the skin. The film forming excipient present in some embodiments can protect the skin. This property will be beneficial, e.g., to patients suffering from post herpetic neuralgia (PHN).

The present invention is also directed to a method of providing anesthesia by administering the sprayable liquid lidocaine solution to the skin. This sprayable liquid solution may be administered to provide local anesthetic to provide pain relief for PHN, or allodynia. Because PHN is a localized condition, localized therapy with a topical analgesic may provide adequate efficacy while mitigating the risk of systemic adverse events compared with oral analgesics (e.g., tricyclic antidepressants, anticonvulsants, opioids).

Existing treatments for PHN are either administered systemically, and so may result in systemic adverse effects, or involve application of a lidocaine patch to already sensitive skin. Moreover, there is a significant delay between application of a lidocaine patch and pain relief. The compositions and methods of the present invention do not have these drawbacks.

In other embodiments of the invention, the sprayable liquid solution may be administered as a local anesthetic to numb an area of skin before an injection or blood draw. In some embodiments, the sprayable liquid solution includes an excipient, such as a dye or colorant, that marks the skin in the area to which the spray is applied so that the healthcare practitioner can easily identify the anesthetized area of skin. In some embodiments, the sprayable liquid solution may be administered as a local anesthetic to the gingiva (gums) or the oral mucosa, e.g., to provide local anesthesia during dental procedures. In some embodiments, the sprayable liquid solution may be administered as a local anesthetic spray to the mucosal lining of the pharynx (throat) for medical procedures that can cause pain in the pharynx, e.g., for pre-operative intubation or prior to an endoscopy. In some embodiments, the sprayable liquid solution is a gel. In some embodiments, the sprayable liquid solution is capable of forming a gel upon contact with the mucosa, meaning that when it is sprayed on mucosal tissue, it becomes a gel.

Other lidocaine sprays contain a low concentration of lidocaine, and/or contain significant amounts of water so do not dry quickly. Moreover, they may not efficiently deliver adequate concentrations of lidocaine with adequate dermal/mucosal penetration to provide sufficient relief for PHN, allodynia, or to provide local anesthesia prior to an injection, dental procedure, intubation or endoscopy.

The concentration of lidocaine in the composition of the invention is around 4 or 5 times higher than the currently available lidocaine spray, and at least eight times more than the currently available lidocaine patch. The sprayable liquid solution of the invention delivers lidocaine more quickly than currently available lidocaine patches, and thus can provide significantly faster pain relief than the patch with significantly less irritation.

After administration of the sprayable liquid solution of the invention, a very thin layer of solution is formed on the skin when this spray is applied. In only a few minutes, e.g., less than 4 minutes, less than 3 minutes, or less than 2 minutes, the solvent/s in the spray evaporate. In some embodiments, the solvent leaves behind a film containing lidocaine. During solvent evaporation, the excipients included in the spray prevent recrystallization of lidocaine and maintain lidocaine in an amorphous state, which is important for its penetration of the skin. This characteristic helps make it possible to incorporate a high concentration of lidocaine in the formulation. Within about 5 minutes, about 4 minutes or about 3 minutes, lidocaine is absorbed into the skin. The skin then acts as a reservoir from which lidocaine is released over time.

In some embodiments, the formulation according to the invention is packaged as a bulk solution containing multiple doses in a pump spray system comprising a sealed container fitted with a spray actuator such as a spray pump, e.g., a metering spray pump.

The pump system may be a pump action spray. Pump action sprays require the application of external pressure for actuation, for example, external manual, mechanical or electrically initiated pressure.

The container holding the solution may be any suitable container for the particular composition, such as a glass, polyethylene, aluminum or steel bottle or canister, or an aluminum pouch within a high density polyethylene bottle. The bottle or canister may be lined with an inert material. The spray pump system may also include a dose indicator or dose counter.

In one embodiment, the invention provides a composition comprising:

a. at least 40% w/w lidocaine;

b. an aliphatic solvent;

c. about 0.1% w/w to about 10% w/w of a washability enhancer; and

d. about 0.1% w/w to about 10% w/w one or more film forming excipients, wherein the film forming excipient (i) has a solubility in water, at a pH between 1 and 10, that requires 30 parts or less of water to dissolve one part of the film forming excipient, (ii) has a solubility in ethanol that requires 30 parts or less of ethanol to dissolve one part of the film forming excipient; and (iii) can prevent crystallization of the lidocaine and maintain the lidocaine in an amorphous state during solvent evaporation when the composition is applied to skin;

wherein the composition is a sprayable liquid solution; and wherein the formulation contains less than 10% w/w water.

In another embodiment, the invention provides a composition comprising:

a. at least 40% w/w lidocaine;

b. about 30% w/w to about 70% w/w of an aliphatic solvent selected from the group consisting of Acetone, Ethanol, Isopropyl Alcohol, and a mixture thereof;

c. about 1% w/w to about 5% w/w of a washability enhancer selected from the group consisting of Dibutyl Sebacate, Triethyl Citrate, Triacetin, Glycerol, Polyethylene Glycol 300, Polyethylene Glycol 400, Polyethylene Glycol 600, Propylene Glycol and a mixture thereof; and

d. about 0.1% w/w to about 10% w/w a film forming excipient, selected from the group consisting of Hydroxypropyl Cellulose, Ethyl Cellulose, Hydroxypropyl Methyl Cellulose, Amino Methacrylate Copolymer, Methacrylic Acid and Methyl Methacrylate Copolymer 1:1, Methacrylic Acid and Methyl Methacrylate Copolymer 1:2, Poly(butylmethacrylate-co-(2-dimethylaminoethyl)methacrylate-co-methyl methacrylate 1:2:1, a Methyl Vinyl Ether-Maleic Anhydride Copolymer, a Butyl Ester of Methyl Vinyl Ether/Maleic Anhydride Copolymer; a Polyvinyl Acetate-Povidone Polymer, and a mixture thereof;

wherein the composition is a sprayable liquid solution; and wherein the formulation comprises less than 10% w/w water.

In other embodiments, the invention provides a composition comprising:

a. at least 40% w/w lidocaine;

b. about 30% w/w to about 70% w/w Ethanol;

c. about 1% w/w to about 5% w/w Polyethylene Glycol 400; and

d. about 0.1% w/w to about 10% w/w a film forming excipient, selected from the group consisting of Hydroxypropyl Cellulose, Ethyl Cellulose, Hydroxypropyl Methyl Cellulose, Amino Methacrylate Copolymer, and a mixture thereof;

wherein the composition is a sprayable liquid solution; and wherein the formulation comprises less than 10% w/w water.

In some embodiments, the composition further comprises one or more penetration enhancers. In some embodiments the composition comprises about 0.1% w/w to about 10% w/w of a penetration enhancer. In other embodiments, the penetration enhancer is selected from the group consisting of Azone, Glycerol Monooleate, Isopropyl Myristate, Octisalate, Oleic Acid, Diethylene Glycol Monoethyl Ether, and a mixture thereof.

In some embodiments, the composition contains a washability enhancer selected from the group consisting of Polyethylene Glycol 300, Polyethylene Glycol 400, Polyethylene Glycol 600, and mixtures thereof.

In some embodiments, the composition may further comprise one or more viscosity increasing agents.

The composition is capable of leaving a film on skin onto which the composition is sprayed after the solvent evaporates.

In some embodiments the composition comprises a unit dose of a volume of about 200 μl or less.

The composition may be in a sealed container fitted with a spray pump.

The invention also provides a method of providing local anesthesia comprising spraying onto a subject's skin a volume of about 200 μL or less of the composition. In some embodiments, the volume of spray administered in a single actuation is about 90 μL. In further embodiments, the single actuation comprises 40 mg lidocaine. In other embodiments, the single actuation comprises 20 mg lidocaine. In some embodiments of the methods, the composition leaves a film on skin after the solvent evaporates.

The invention also provides a method of providing local anesthesia to a subject having post-herpetic neuralgia, comprising spraying the composition of claim 1 on an area of the subject's skin. In addition, the invention provides a method of providing local anesthesia to a subject having allodynia, comprising spraying the composition on an area of the subject's skin. Furthermore, the invention provides a method of providing anesthesia to an injection area of the skin prior to an injection, comprising spraying the composition on an area of the subject's skin.

In yet another embodiment, the invention provides a composition comprising:

a. at least 40% w/w lidocaine;

b. an aliphatic solvent;

c. about 0.1% w/w to about 10% w/w of a penetration enhancer;

d. about 0.1% w/w to about 5% w/w of a plasticizer; and

e. a viscosity increasing agent;

wherein the composition is a sprayable liquid solution; and wherein the formulation contains less than 10% w/w water.

In some embodiments, the penetration enhancer is selected from the group consisting of Azone, Glycerol Monooleate, Isopropyl Myristate, Octisalate, Oleic Acid, Diethylene Glycol Monoethyl Ether, and a mixture thereof.

In some embodiments, the composition leaves a film on skin onto which the composition is sprayed after the solvent evaporates. In some embodiments the composition comprises a unit dose of a volume of about 200 μl or less. The composition may be in a sealed container fitted with a spray pump.

The invention also provides a method of providing local anesthesia comprising spraying onto a subject's skin a volume of about 200 μL or less of the composition. In some embodiments, the invention provides a method of providing local anesthesia to a subject comprising spraying onto an area of pharynx mucosal lining of the subject a volume of about 200 μL or less of the composition. In some embodiments, the method is performed to prevent pain during an intubation or endoscopic procedure. In some embodiments, the volume of spray administered in a single actuation is about 90 μL. In further embodiments, the volume of spray administered comprises about 40 mg lidocaine. In other embodiments, the volume of spray administered comprises about 20 mg lidocaine. In some embodiments of the methods, the composition leaves a film on skin after the solvent evaporates.

In yet another embodiment, the invention provides a composition comprising:

a. about 2% w/w to about 50% w/w w/w lidocaine;

b. an aliphatic solvent;

c. about 0.1% w/w to about 10% w/w of a penetration enhancer;

d. about 0.1% w/w to about 5% w/w of a plasticizer;

e. a viscosity increasing agent; and

f. about 0.1% w/w to about 2% w/w of a flavoring agent;

wherein the composition is a sprayable liquid, and wherein the formulation contains less than 10% w/w water. In further embodiments, this sprayable liquid is a gel. In some embodiments, this sprayable liquid forms a gel upon contact with mucosal tissue.

In some embodiments, the viscosity increasing agent is a bioadhesive viscosity increasing agent. In some embodiments, the composition comprises a unit dose of a volume of about 200 μl or less.

The invention also provides a method of providing local anesthesia to a subject comprising spraying onto an area of gingiva or oral mucosa of the subject a volume of about 200 μL or less of the composition. In some embodiments of the method, the volume of spray administered in a single actuation is about 50 μL. In some embodiments, the volume of spray administered in a single actuation comprises about 2 mg to about 40 mg lidocaine. In other embodiments, the single actuation comprises 20 mg lidocaine.

For most applications, the composition of the invention comprises at least about 40% w/w lidocaine, and contains one or more solvents, and may also contain one or more film forming excipients, penetration enhancers, and/or viscosity increasing agents. For treatment requiring application to the oral mucosa or gingiva the composition of the invention comprises about 2% to about 50% w/w lidocaine, and contains one or more solvents, and may also contain one or more film forming excipients, penetration enhancers, and/or viscosity increasing agents.

In some embodiments, the composition is free of water, or contains less than 5% w/w water, or less than 1% w/w water.

The composition contains lidocaine, which is chemically designated as acetamide, 2-(diethylamino)-N-(2,6-dimethylphenyl). The composition may contain about 2% w/w to about 50% w/w, about 10% w/w to about 50% w/w, about 2% w/w to about 10% w/w, about 40% w/w to about 70% w/w, about 45% w/w to about 60% w/w, about 40% w/w to about 50% w/w, about 40% w/w, about 41% w/w, about 42% w/w, about 43% w/w, about 44% w/w, about 45% w/w, about 46% w/w, about 47% w/w, about 48% w/w, about 49% w/w, about 50% w/w, about 55% w/w, or about 60% w/w lidocaine. The composition may contain more than: 40% w/w, 41% w/w, 42% w/w, 43% w/w, 44% w/w, 45%, w/w, 46% w/w, 47% w/w, 48% w/w, 49% w/w, or 50% w/w lidocaine.

The composition may also contain one or more aliphatic solvents such as Acetone, Ethanol, and/or Isopropyl Alcohol. The total aliphatic solvent concentration may be around 20% w/w to about 98% w/w of the composition, but generally will make up the remainder of the weight of the composition and is a sufficient quantity to dissolve the other ingredients. In some embodiments of the invention, the concentration of aliphatic solvent is about 30% w/w to about 95% w/w, about 80% w/w to about 95% w/w, or about 30% w/w to about 70% w/w, about 40% w/w to about 60% w/w, about 45% to about 55%, about 50% w/w to about 55% w/w, or about 70% w/w to about 98% w/w.

The composition may also contain one or more penetration enhancers such as Azone, Glycerol Monooleate, Isopropyl Myristate, Octisalate, Oleic Acid, and/or Diethylene Glycol Monoethyl Ether (such as Transcutol® or Transcutol® P). In some embodiments of the invention, the concentration of the one or more penetration enhancers in the composition is about 0.1% w/w to about 20% w/w, about 0.1% w/w to about 15% w/w, about 0.1% w/w to about 10% w/w, about 0.1% w/w to about 5% w/w, or about 0.5% w/w to about 8%.

In some embodiments, particularly for compositions intended for application to the skin, the composition contains a film forming excipient. The film forming excipient is an excipient, preferably a polymer, that is soluble in aliphatic solvents such as ethanol or a mixture of ethanol with other aliphatic solvents.

The film forming excipient is also soluble in aqueous solutions, preferably water. Although, for film forming excipients having pH dependent solubility, the pH of the aqueous solution must be above or below the specific trigger pH for that excipient to dissolve. For example, some of these film forming excipients dissolve in aqueous solutions only above pH 6.0, only above pH 7.0, or only below pH 5.0. Therefore, soluble in water is defined herein to mean that the film forming excipient has a solubility in water, at the pH required to dissolve that excipient, that requires 30 parts or less of water (solvent) to dissolve one part of the film forming excipient (solute), or requires 10 parts of water or less to dissolve one part of the film forming excipient. Thus, the film forming excipient has a solubility in water, at a pH between 1 and 10, that requires 30 parts or less of water (solvent) to dissolve one part of the film forming excipient (solute). For example, the film forming excipient has a solubility requiring 30 parts or less of water (solvent) to dissolve one part of the film forming excipient (solute), when the pH of the water is selected from the group consisting of: below pH 5.0, above pH 4, above pH 5, above pH 5.5, above pH 6, and above pH 7.

In addition, the film forming excipient may have a solubility in ethanol that requires 30 parts or less of ethanol (solvent) to dissolve one part of the film forming excipient (solute), or requires 10 parts or less of ethanol to dissolve one part of the film forming excipient. Solubility is evaluated at room temperature.

Where the film forming excipient is a mixture of excipients, the mixture should have the desired characteristics described above.

Moreover, the film forming excipient, or mixture of film forming excipient with other excipients such as a plasticizer or washability enhancer, prevents recrystallization of lidocaine and maintains lidocaine in an amorphous state during and after solvent evaporation. This characteristic can be evaluated, for example, by the crystallization experiment described in Example 3, or indirectly by the skin permeation and penetration experiments described in Example 4.

Examples of film forming excipients that have the desired characteristics include Hydroxypropyl Cellulose (HPC or Hypromellose), Ethyl Cellulose, Hydroxypropyl Methyl Cellulose (HPMC), Eudragit® E100 (Amino Methacrylate Copolymer), Eudragit® L100 (Methacrylic Acid and Methyl Methacrylate Copolymer 1:1) (which dissolves above pH 6.0), Eudragit® 5100 (Methacrylic Acid and Methyl Methacrylate Copolymer 1:2) (which dissolves above pH 7.0), Eudragit® E100 (Poly(butylmethacrylate-co-(2-dimethylaminoethyl)methacrylate-co-methyl methacrylate 1:2:1) (which dissolves below pH 5.0), Methyl Vinyl Ether-Maleic Anhydride Copolymers such as Gantrez® ES-435, a Butyl Ester of Methyl Vinyl Ether/Maleic Anhydride Copolymer; and Polyvinyl Acetate-Povidone Polymer.

The total concentration of the one or more film forming excipients in the composition may be about 0.1% w/w to about 20% w/w, about 0.1% w/w to about 10% w/w, about 0.5% w/w to about 8% w/w, about 0.5% w/w to about 6% w/w, about 0.5% w/w to about 5% w/w, about 0.5% w/w to about 2% w/w, e.g., it may be about 0.8%, about 0.9% w/w, about 1% w/w, about 2% w/w, about 3% w/w, about 4% w/w, about 5% w/w or about 6% w/w.

In some embodiments, after application of the composition, a barrier film is formed within about 5 minutes or less of application. A washability enhancer may be included in the composition to make the film more washable, i.e., easier to remove from skin with water or soap and water, particularly when the film forming excipient is an excipient that requires a certain pH to dissolve in water or an aqueous environment, such as Eudragit® S100. In some cases, the washability enhancer also acts as a plasticizer. The washability enhancer may be, e.g., propylene glycol, glycerin, or a low molecular weight polyethylene glycol (PEG) containing two-to-four ethylene glycol units per polymer unit, such as polyethylene glycol 300, 400 or 600.

The concentration of washability enhancer in the composition may be about 1% to about 300% of the weight of the film forming excipient in the composition. In some embodiments it is about 50% to about 250% of the weight of the film forming excipient in the composition. In other embodiments, it is about 20% to about 100% of the weight of the film forming excipient in the composition. It may also be about 40%, about 45%, about 50%, about 55%, about 60%, about 70%, about 80%, about 90%, about 100%, about 120%, about 140%, about 160%, about 180%, about 190%, about 200%, about 210%, about 220%, about 230%, about 240%, or about 250% of the weight of the film forming excipient in the composition. With regard to the total weight of the composition, in some embodiments, the concentration of washability enhancer in the composition may be about 0.1% w/w to about 20% w/w, about 0.1% w/w to about 15% w/w, about 0.1% w/w to about 10% w/w, about 0.1% w/w to about 5% w/w, about 0.5% w/w to about 5% w/w, or about 1% w/w to about 5% w/w of the composition.

The composition may include one or more plasticizers such as Dibutyl Sebacate, Triethyl Citrate, Triacetin, Glycerol, a low molecular weight Polyethylene Glycol, e.g., Polyethylene Glycol 300, 400 or 600, and/or Propylene Glycol. The concentration of plasticizer in the composition may be about 0.1% w/w to about 20% w/w, about 0.1% w/w to about 15% w/w, about 0.1% w/w to about 10% w/w, about 0.1% w/w to about 5% w/w, about 0.5% w/w to about 5% w/w, or about 1% w/w to about 5% w/w of the composition. In some embodiments it is about 0.5% w/w, about 1% w/w, about 1.5% w/w, about 2% w/w, about 2.5% w/w, about 3% w/w, about 3.5% w/w, or about 4% w/w, of the composition. In some embodiments, the plasticizer is also a washability enhancer.

The composition may also include one or more viscosity increasing agents, e.g., Povidone, Glycerin, Hydroxypropyl Cellulose, Methylcellulose, and/or Carboxymethylcellulose. The concentration of the viscosity increasing agent, if included, may be about 0.1% w/w to about 10% w/w, about 0.1% w/w to about 8% w/w, or about 0.1% w/w to about 5% w/w of the composition, e.g., about 0.5% w/w, about 1% w/w, about 1.5% w/w, about 2% w/w, or about 2.5% w/w of the composition.

In some embodiments, the viscosity increasing agent is bioadhesive. Such agents include Carbopol®. Carbopols® are high molecular weight, crosslinked polyacrylic acid polymers. The polymers differ by crosslink density and can be grouped into the following categories: (1) Carbopol® homopolymers, such as acrylic acid crosslinked with allyl sucrose or allyl pentaerythritol, e.g., Carbopols® 71G NF (viscosity 4,000-11,000), 971P NF (viscosity 4,000-11,000), 974P NF, (viscosity 29,400-39,400), 980 NF (viscosity 40,000-60,000), 981 NF (viscosity 4000-10,000), 5984 EP (viscosity 30,500-39,400), 934 NF (viscosity 30,500-39,400), 934P NF (viscosity 29,400-39,400), 940 NF (viscosity 40,000-60,000), 941 NF (viscosity 4,000-10,000); and (2) Carbopol® copolymers: acrylic acid and C10-C30 alkyl acrylate crosslinked with allyl pentaerythritol, e.g., Carbopol® 1342 NF, viscosity 9,500-26,500.

The composition of the present invention may also include other formulation excipients, added, e.g., to achieve a desired consistency or appearance, or to protect the formulation components from degradation and oxidation. Such excipients include, for example, cosolvents, stabilizing agents, antioxidants, humectants, preservatives, pH modifiers, flavoring agents, colorant, dye, and fragrances known in the art of formulation.

For example, compositions intended for application to the gingiva or oral mucosa may contain one or more flavoring agents. The flavoring agent may be, for example, bubble gum flavor, peppermint flavor, cherry flavor, grape flavor, and orange flavor. The flavoring agent may also be a mixture of two or more agents if they are compatible. The concentration of the flavoring agent, if included, may be about 0.1% w/w to about 5% w/w, about 0.1% w/w to about 3% w/w, or about 0.1% w/w to about 2% w/w of the composition, e.g., about 0.5% w/w, about 1% w/w, about 1.5% w/w, about 2% w/w, or about 2.5% w/w of the composition.

Another optional ingredient is a pH modifier, such as tromethamine. The concentration of the pH modifier may be about 0.05 g/L to about 0.2 g/L. Here, grams per liter (g/L) refers to grams of organic proton acceptor per liter aliphatic solvent in the composition. In some embodiments, the concentration of the pH modifier is about 0.05 g/L, about 0.1 g/L, or about 0.2 g/L. In some embodiments of the invention the aliphatic solvent contains Tromethamine.

In some embodiments, particularly where the composition is intended as a local anesthetic to numb an area of skin before an injection or blood draw, the composition may include a dye or colorant to mark the skin where the spray is applied so that the healthcare practitioner can easily identify the anesthetized area of skin.

In some embodiments, particularly for application to the skin, the composition of the present invention may contain a fragrance or perfume to impart a desired aroma, or to mask odors that may be associated with other components of the composition. If a fragrance is included, the concentration may be about 0.01% w/w to about 5% w/w of the composition, or about 0.1% w/w to about 1% w/w. The fragrance may also act as a solvent, and/or may be aqueous to avoid irritating the skin.

Any fragrance suitable for application to the skin can be used herein including a wide variety of fragrances and perfumes that are known to those skilled in the art. The particular perfume used is largely a matter of choice, however, the fragrance should be used at a level effective for providing a noticeable aroma to the composition, or for masking undesired aroma of the composition. Also, the fragrance and whatever carriers accompany it should not impart excessive stinging to the skin, especially broken or irritated skin.

Typical fragrances are described in Arctander, Perfume and Flavour Chemicals (Aroma Chemicals), Vol. I and II (1969, Allured Publishing Corporation, 1969 (ISBN 0931710375, 9780931710377), and Arctander, Perfume and Flavor Materials of Natural Origin (1994, by Allured Pub Corp) (ISBN 0931710367; ISBN13: 9780931710360). Fragrance used in the present invention may also contain solubilizers, diluents, or solvents which are well known in the art.

The excipients may serve more than one function in some embodiments of the sprayable solution. For example, an excipient may function as both a film forming agent and a viscosity increasing agent, or both a plasticizer and washability enhancer.

The composition is a liquid at room temperature and is sprayable, meaning that the viscosity of the composition allows it to be applied using a spray pump at room temperature. In one embodiment, the composition is contained in a container comprising a spray pump with metering valve. For some applications, e.g., administration to the oral mucosa or gingiva, the container may have a stem nozzle. In one embodiment, the viscosity of the composition is less than about 50 cPs, less than about 20 cPs, or less than about 10 cPs. For example, the viscosity of the composition may be about 4 cPs, about 5 cPs, about 6 cPs, about 7 cPs, about 8 cPs, or about 9 cPs. Viscosity is measured using a viscometer such as a Brookfield Viscometer.

The invention also provides a method of providing local anesthesia, by spraying the composition on the skin, gingiva, oral mucosa or mucosal lining of the pharynx of a subject, such as a human. In an embodiment of this method, the amount of lidocaine administered in a single application is about 10 to about 100 mg, about 10 mg to about 80 mg, about 15 mg to about 75 mg, about 15 mg to about 60 mg, about 20 mg to about 60 mg, about 20 mg to about 50 mg, about 20 mg to about 40 mg, about 1 mg to about 50 mg, or about 2 mg to about 40 mg. In some embodiments, the amount of lidocaine administered in a single application is about 10 mg, about 15 mg, about 20 mg, about 25 mg, about 30 mg, about 35 mg, about 40 mg, about 45 mg, about 50 mg, about 55 mg, about 60 mg, or about 65 mg. This amount of lidocaine can be delivered in a single actuation or in more than one actuation. For example, the desired dose of lidocaine may be administered in a single actuation of the spray containing about 40 mg, or in two actuations each containing about 20 mg, or even in more actuations of smaller doses that add up to a total of at least about 40 mg.

In some embodiments of the invention, the composition is administered one to four times per 24 hour period. In other embodiments, the composition is administered one to eight times per 24 hour period. In one embodiment the composition is administered four times per 24 hour period. In some embodiments, a single administration can consist of two spray actuations, such as where each actuation delivers less than about 40 mg of lidocaine, e.g., where each actuation delivers about 20 mg of lidocaine.

In some embodiments, the volume of spray administered is about 500 μL or less, about 400 μL or less, about 300 μL or less, about 200 μL or less, about 150 μL or less, about 100 μL or less, about 90 μL or less, about 80 μL or less, or about 70 μL or less. The volume of spray administered may be, e.g., about 500 μL, about 400 μL, about 300 μL, about 200 uL, about 150 μL, about 100 μL, about 95 μL, about 90 μL, about 85 μL, about 80 μL, about 75 μL, about 70 μL, about 65 μL, about 60 μL, about 55 μL, about 50 μL, about 45 μL, or about 40 μL. It is possible to dissolve 40 mg or more of lidocaine in a volume less than 100 μL of the composition of the invention, and achieve a lidocaine concentration of 40% w/w or higher.

In further embodiments, the invention provides any of the compositions described herein for use as a medicament, for use in providing local anesthesia, for use in providing local anesthesia to a subject having post-herpetic neuralgia, for use in providing local anesthesia to a subject having allodynia, for use in providing anesthesia to an injection area of the skin prior to an injection, for use in providing local anesthesia to an area of gingiva or oral mucosa, or for use in providing local anesthesia to an area of pharynx mucosal lining.

### Definitions

As used herein, the following words and phrases are generally intended to have the meanings as set forth below, except to the extent that the context in which they are used indicates otherwise.

As used in the specification and the appended claims, topical means that the formulation is applied to a particular place on or in the body. Most often topical administration means application to body surfaces such as the skin or mucous membranes. Many topical medications are epicutaneous, meaning that they are applied directly to the skin. Topical medications may also be inhalational, such as asthma medications, or applied to the surface of tissues other than the skin, such as eye drops applied to the conjunctiva, or ear drops placed in the ear, or medications applied to the surface of a tooth.

The term “subject,” as used herein, refers to a human, e.g., a human patient.

The singular forms “a,” “an,” and “the” include plural reference unless the context clearly dictates otherwise. Thus “a” or “an” can mean one or more.

The term “about” as used herein means approximately ±10%. When the term “about” is used in conjunction with a numerical value or range, it modifies that value or range by extending the boundaries above and below the numerical values set forth. In general, the term “about” is used herein to modify a numerical value above and below the stated value by a variance of 10 percent, up or down (higher or lower), i.e., ±10%, unless a different variance is indicated (e.g., ±30%, ±20%, ±5%, ±1%, etc.).

Also as used herein, “and/or” refers to and encompasses any and all possible combinations of one or more of the associated listed items, as well as the lack of combinations when interpreted in the alternative (“or”). Also, to the extent that the terms “in” or “into” are used in the specification or the claims, it is intended to additionally mean “on” or “onto.”

As used herein, unless otherwise specified all formulation ingredient percentages are described as percent weight by weight (% w/w), which refers to the weight of an ingredient as a percentage of the total composition weight.

As used herein, the term “room temperature” refers to a comfortable ambient temperature, generally taken as 20-25° C., preferably 25° C.

An “effective amount” is the quantity of compound or formulation in which a beneficial clinical outcome can be achieved when the compound or formulation is administered to a subject suffering from or at risk of suffering from a condition to be treated. A “beneficial clinical outcome” can include one or more of: a reduction in pain, or in frequency of painful episodes.

Wherever aspects or features are described herein with the language “comprising,” otherwise analogous aspects or features described in terms of “consisting of” and/or “consisting essentially of” are also provided. To the extent that the term “includes” or “including” is used in the specification or the claims, it is intended to be inclusive in a manner similar to the term “comprising” as that term is interpreted when employed as a transitional word in a claim.

As used herein, the terms “optional” and “optionally” mean that the subsequently described circumstance may or may not occur, so that the description includes instances where the circumstance occurs and instances where it does not.

In addition, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, and the like. As a non-limiting example, each range discussed herein can be readily broken down into a lower third, middle third and upper third, and the like. As will also be understood by one skilled in the art all language such as “up to,” “at least,” “greater than,” “less than,” include the number recited and refer to ranges which can be subsequently broken down into sub-ranges as discussed above. Finally, as will be understood by one skilled in the art, a range includes each individual member. For example, a group having 1-3 members refers to groups having 1, 2, or 3 members. Similarly, a group having 1-5 members refers to groups having 1, 2, 3, 4, or 5 members, and so forth.

Several embodiments of the invention are described herein. In addition, the invention is described with reference to the following examples. This invention is not limited to the embodiments or examples described herein. Modifications and variations may suggest themselves and are intended to be within the scope of the appended claims.

### EXAMPLES

### Example 1: Sprayable Liquid Lidocaine Solutions with Film Forming Excipient

The following tables describe some exemplary compositions for the sprayable liquid lidocaine solution. These formulations may be administered, for example, to provide local anesthesia or pain relief to subjects suffering from PHN or allodynia, to provide pain relief or local anesthesia to the gingiva or oral mucosa, to provide anesthesia to an area of the gingiva prior to a dental procedure, to provide anesthesia to an injection area of the skin prior to an injection, or to prevent pain during an endoscopy or intubation. A manufacturing process for these formulations is described below the tables.

TABLE 1

Composition No.

| | 1 | 2 |
|---|---|---|
| Ingredients | % w/w | % w/w |
| Lidocaine | 42 | 50 |
| PEG 400 | 2 | 2 |
| Diethylene glycol monoethyl ether | 4 | 4 |
| Hydroxy propyl cellulose | 1 | 1 |
| Ethanol | 51 | 43 |
| Total | 100 | 100 |

### Manufacturing Process:

Ethanol is added to a suitable vessel. Hydroxy propyl cellulose and polyethylene glycol 400 are added, and these ingredients are stirred until a clear solution is formed. Diethylene glycol monoethyl ether is added and the solution is mixed further. Lidocaine is added and the mixture is stirred until a clear solution is achieved. The solution is filled into a suitable container and a spray pump is affixed to the container.

TABLE 2

| | Composition No. |
|---|---|
| | 3 |
| Ingredients | % w/w |
| Lidocaine | 45 |
| PEG 400 | 2 |
| Amino Methacrylate Copolymer | 4 |
| Hydroxy propyl cellulose | 1 |
| Ethanol | 48 |
| Total | 100 |

### Manufacturing Process:

Ethanol is added to a suitable vessel. Hydroxy propyl cellulose and polyethylene glycol 400 are added, and these ingredients are stirred until a clear solution is formed. Amino methacrylate copolymer is added and the solution is mixed further. Lidocaine is added and the mixture is stirred until a clear solution is achieved. The solution is filled into a suitable container and a spray pump is affixed to the container.

### Example 2: Sprayable Liquid Lidocaine Solutions

The following tables describe some exemplary compositions for the sprayable liquid lidocaine solution. While each formulation in this table is described for a particular application, each formulation may also be administered for other applications. A manufacturing process for these formulations is described below the table.

TABLE 3

| | Spray Formulation | | | | | |
|---|---|---|---|---|---|---|
| | | | | | | Pain during |
| | | | | Pain at | | endoscopies |
| | Neuropathic | Neuropathic | Neuropathic | injection | Dental | and |
| | Pain | Pain | Pain | site | anesthesia | intubations |
| | Composition No. | | | | | |
| | 4 | 5 | 6 | 7 | 8 | 9 |
| | Mg % | Mg % | Mg % | Mg % | Mg % | Mg % |
| | w/w | w/w | w/w | w/w | w/w | w/w |
| Lidocaine | 45 mg | 45 mg | 45 mg | 45 mg | 40 mg | 35 mg |
| | 42.9% | 42.9% | 42.9% | 42.9% | 40% | 47.9% |
| Polyethylene | 2 mg | 2 mg | 2 mg | 2 mg | 2 mg | 2 mg |
| Glycol 400 | 1.9% | 1.9% | 1.9% | 1.9% | 2% | 2.7% |
| Diethylene | 4 mg | 4 mg | 4 mg | 4 mg | 1 mg | 4 mg |
| glycol | 3.8% | 3.8% | 3.8% | 3.8% | 1% | 5.5% |
| monoethyl ether | | | | | | |
| Ethanol | 53 mg | 53 mg | 53 mg | 54 mg | 55.8 mg | 27 mg |
| | 50.5% | 50.5% | 50.5% | 51.4% | 55.8% | 37% |
| Amino | — | 1 mg | | — | — | — |
| Methacrylate | | 0.9% | | | | |
| Copolymer | | | | | | |
| Carbopol ® 974 | — | | | — | 0.2 mg | — |
| | | | | | 0.2% | |
| Flavoring Agent | — | | | — | 1 mg | — |
| | | | | | 1% | |
| Hydroxy propyl | — | | 1 mg | — | — | — |
| methyl cellulose | | | 0.9% | | | |
| Hydroxy propyl | 1 mg | | | — | — | 5 mg |
| cellulose | 0.9% | | | | | 6.9% |
| Total | 105 mg | 105 mg | 105 mg | 105 mg | 100 mg | 73 mg |
| | 100% | 100% | 100% | 100% | 100% | 100% |

### Manufacturing Process:

Ethanol is added to a suitable vessel. Polyethylene glycol 400 is added to this vessel. and stirred until a clear solution is formed. If the formulation contains Hydroxy propyl cellulose or hydroxy propyl methyl cellulose or Amino Methacrylate Copolymer, they are added, and the ingredients are stirred until a clear solution is formed. Diethylene glycol monoethyl ether is added and the solution is stirred further. If the composition contains a flavoring agent, it is added and the solution is stirred further. If the composition contains a Carbopol®, it is added and the solution is stirred further until a clear solution is formed. Lidocaine is added and the mixture is stirred until a clear solution is achieved. The solution is filled into a suitable container and a spray pump is affixed to the container.

### Example 3: Crystallization Study

This study is performed to evaluate whether lidocaine crystallized from the film forming formulation following evaporation of the formulation solvent on a glass substrate, and to determine whether the film forming excipient or washability enhancer impact the crystallization of lidocaine.

A Hamilton syringe is used to spray 10 μL of each formulation to be tested onto a 0.8 cm² silicon wafer plate. Each plate is then stored for 20 minutes RT (22° C.)/˜50% RH (ambient RH) before samples were taken.

Samples are collected at the edge and center of the plate using a spatula. The samples are evaluated by Two Dimensional powder X-ray diffractometry (2DPXRD). The instrument used is a D8 Discover, Bruker 2D X-ray diffractometer. Cobalt is used as the source and the collimator setting was 800 microns beam diameter. The lidocaine crystal structures are retrieved from CSD and the obtained XRD patterns are compared with reported CSD crystal structures.

### Example 4A: In Vitro Permeation Testing

A comparison of skin permeation by the formulations in Table 4 was performed using a Franz Cell Diffusion Apparatus.

Human skin from cosmetic reduction surgery was used. Subcutaneous fat was removed mechanically and the skin was dermatomed to a thickness of about 400 Healthy dermatomed skin was mounted between the two halves of a Franz cell.

The Stratum corneum was removed from the dermatomed human skin by tape stripping. Following tape stripping, the epidermis was separated from the dermis by dry heating.

In vitro skin permeation was investigated using 100% PBS as receiver fluid. Franz diffusion cells, previously calibrated, with an average surface area of approximately 0.6 cm² and a volume of approximately 2.0 mL were employed.

The integrity of the skin was assessed before dosing. Dermatomed skin from one donor only was mounted between the donor and receiver compartments and the cells were sealed together using Parafilm® and clips.

The donor and receiver chambers were filled with PBS solution and a small magnetic follower was placed in the receiver compartment.

Cells were equilibrated in a water bath ensuring a membrane temperature of 32° C. for 30 min (water bath temperature of 37° C.). The resistance of the skin in each Franz cell was measured using the LCR 6401 Databridge. The electrodes were placed in the receiver compartment through the sampling arm and the donor chamber. The LCR was set at 100 Hz and set to ‘R’ for resistance. Cells with a resistance below the acceptable limits were discarded and remounted. Acceptable limits are defined according to the measurement of controls for dermatomed skin, where the skin has been deliberately perturbed. Cells with greater than twice the resistance (KΩ) of the control were considered acceptable and selected for the Franz cell permeation experiment.

Following skin integrity testing, the PBS solution was removed from each compartment and the receiver compartment of acceptable cells was filled with receiver fluid (100% PBS). Each cell was then equilibrated to ensure a surface temperature of 32° C. (external skin surface temperature) for at least 30 min prior to dosing (water bath temperature of 37° C.).

A positive displacement pipette was used to apply the lidocaine compositions (about 6 mg) described in Table 4 directly onto the skin (10 mg/m2).

Receiver fluid (200 μL) was removed at time points t=0, 4, 8, and 24 h and transferred to a HPLC and analyzed. Fresh pre-warmed receiver fluid (200 μL) was used to replace the receiver fluid removed at each time point.

TABLE 4
Test formulations of Lidocaine spray

| | Solvent system and level of excipient (% w/w) | | | | | |
|---|---|---|---|---|---|---|
| | | | | | | Lidoderm |
| Excipient | MS04 | MT01v2 | MS10 | MS12 | MT02 | Patch (5%) |
| Ethanol | 63.72 | 22.17 | 44.03 | 53.00 | 26.13 | |
| Water | 24.89 | 39.94 | 29.34 | — | 47.00 | |
| Propylene glycol | — | — | — | — | 10.44 | |
| PEG-400 | — | 4.51 | — | 2.00 | 5.25 | |
| Transcutol P | — | 22.13 | 1.83 | 4.00 | — | |
| Benzyl alcohol | 2.90 | — | 1.83 | — | — | |
| Eudragit E100 | 2.93 | — | 1.83 | — | — | |
| Hydroxypropyl-cellulose | | | | 1.0 | | |
| Lidocaine | 5.56 | 11.26 | 21.13 | 40.00 | 11.18 | |

### Example 4B: In Vitro Skin Penetration Experiment

Following completion of the permeation experiment above, quantification of lidocaine on the surface and within the Stratum corneum, epidermal membrane and partial dermis was performed.

1. Recovery of Lidocaine from the Surface of the Skin

Lidocaine was recovered from the surface of the skin using the following procedure:
(i) Three cotton swabs were used to remove lidocaine from the surface of the skin.
(ii) After dismantling the donor chamber from the Franz cell one of the dry cotton wool swabs was used to remove all of lidocaine from the surface of the skin and the swab placed into a 7 mL glass vial.
(iii) A second swab was then immersed into the extraction solvent (90:10 v/v acetonitrile:water, Section) and used to swab the surface of the skin; this swab was then placed into the vial containing the first swab.
(iv) The final swab was used dry to swab the surface of the skin and then placed into the glass vial containing the two other swabs.
(v) An initial tape strip from the surface of the skin was removed and placed into the vial from Step (iv).
(vi) Extraction diluent (90:10 v/v acetonitrile:water, 2 mL) was added to the vial from Step (v).
(vii) The vials were shaken on an orbital shaker at ambient temperature for 16-20 h.
(viii) Following the extraction procedure, the extraction solvent system was removed from the vials and centrifuged to remove un-dissolved materials and particulates.
(ix) The supernatant was analyzed by HPLC. The amounts of lidocaine (% dose) recovered from each layer, as well as the remaining skin, were calculated by comparison to a standard of known lidocaine concentration.

| | HPLC-UV/Vis | | |
|---|---|---|---|
| | (Quaternary capabilities, P_{MAX} ≥ 5000 Psi/345 Bar/35 MPa) Empower | | |
| | 3 Chromatography Data Software | | |
| Column | Zorbax SB-C8 (4.6 × 250 mm, 5 μm particle size) | | |
| Detection | 215 nm | | |
| Sample Temperature | 25° C. | | |
| Column Temperature | 45° C. | | |
| Flow Rate | 1 mL/min | | |
| Mobile Phase | Mobile phase A - 0.05M phosphoric acid Mobile phase B - acetonitrile | | |
| | Time (min) | Mobile phase A (%) | Mobile phase B (%) |
| Flow table | 0 | 85 | 15 |
| | 2 | 85 | 15 |
| | 10 | 25 | 75 |
| | 11 | 85 | 15 |
| | 14 | 85 | 15 |

| | |
|---|---|
| Sample and standard diluent | 50:50 water:methanol |
| Injection Volume | 5 μL |
| Run Time | 14 min |
| Needle wash | 60:40 ACN:water |
| Seal wash | 60:40 ACN:water |

2. Recovery of Lidocaine from the Stratum Corneum

Lidocaine was recovered from the Stratum corneum of the skin using the following procedure:
(i) Approximately 5 tape strips were removed from the surface of the skin to separate the S. corneum from the epidermis and were placed in a 7 ml glass vial.
(ii) Extraction diluent (90:10 v/v acetonitrile:water, 2 mL) was added to the vial from Step (i).
(iii) Steps (vii)-(ix) described in Section 1 above, were performed to complete the recovery of lidocaine from the S. corneum.

3. Recovery of Lidocaine from the Epidermis and Partial Dermis

The remaining epidermis and partial dermis was processed as follows:
(i) The epidermis and partial dermis was separated using the method described above and placed into individual tissue homogenizer vials.
(ii) Using a grade-A glass bulb pipette 1 mL of extraction diluent (90:10 v/v acetonitrile:water) was added to the tissue homogenizer vials.
(iii) The contents of the tissue homogenizer vials from Step (ii) were homogenized at ambient temperature.
(iv) The contents of the tissue homogenizer vials from Step (iii) were emptied into a 7 mL glass vial.
(v) Extraction diluent (90:10 v/v acetonitrile:water, 1 mL) was added to the empty tissue homogenizer vial from Step (iv) and the vial was vortex mixed for ca. 30 seconds and the contents then emptied into the glass vial from Step (v).
(vi) Steps (vii)-(ix) from Section 1 were performed to complete the recovery of lidocaine from the epidermis and partial dermis.

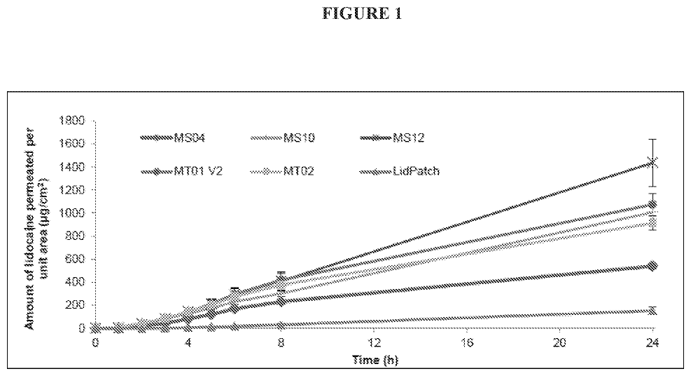

FIG. 1 shows the cumulative amount of lidocaine in the Franz Cell receiver compartment per unit area (μg/cm²) from representative formulations over the 24 h experimental period.

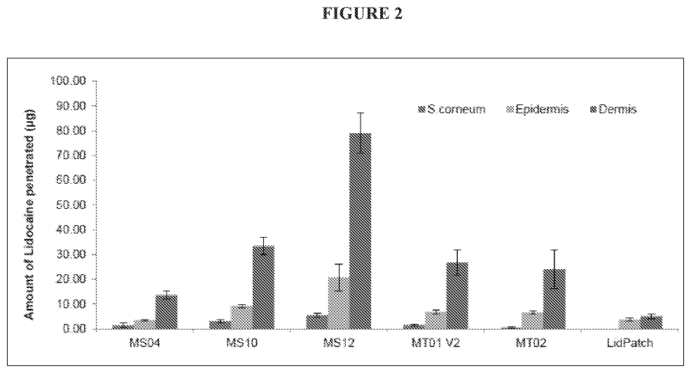

FIG. 2 depicts the amount of lidocaine (μg) penetrated into the S. corneum, epidermis and partial dermis following application of formulations outlined in Table 4 and Lidoderm patch over the 24 h experimental period. Each point represents the mean amount±SEM (n=5-6).

The data in the table below is for the flux of lidocaine across human skin following application of the test formulations from Table 4, calculated between 3-8 h. The data represents the mean±SEM (n=5-6).

TABLE 5

| | | Flux of lidocaine | |
|---|---|---|---|
| Solvent | Approximate drug | between 3-8 h | Rank |
| system/Formulation | loading (% w/w) | (μg/cm²/h) | Order |
| MS12 | 40.0 | 251.29 ± 33.40 | 1 |
| MT01v2 | 12.5 | 183.35 ± 14.42 | 2 |
| MS10 | 25.0 | 170.64 ± 15.23 | 3 |
| MT02 | 12.5 | 151.48 ± 7.91 | 4 |
| MS04 | 5.0 | 90.27 ± 3.29 | 5 |
| Lidoderm (comparator) | 5.0 | 32.40 ± 5.39 | 6 |

Comparative in vitro permeability tests show that Lidocaine from the spray formulation of the invention is absorbed almost 8 times more efficiently than lidocaine from LIDODERM® patch, enabling less drug in a smaller volume of the spray to be applied to achieve the same effect as the patch. Unlike the patch, the lidocaine is absorbed from the spray within 30 seconds, the absorption from the patch is around 45 minutes (Lidoderm PI), hence pain relief from the spray is much faster. Once absorbed in the skin, the skin acts as a depot from which the drug is released over 12 hours in a manner similar to the patch. Patches have issues of adherence throughout 12 hours, and it is also painful to remove these large patches before applying a new patch to skin which has become sensitive. Since the film formed from the spray is breathable and easily washed off soon after the drug has been absorbed, the integrity of the skin is maintained unlike a patch where sensitization and irritation issues may occur.

What is claimed is:

1. A composition comprising:
a. at least 40% w/w lidocaine;
b. an aliphatic solvent;
c. about 0.1% w/w to about 10% w/w of a washability enhancer; and
d. about 0.1% w/w to about 10% w/w one or more film forming excipients, wherein said film forming excipient (i) has a solubility in water, at a pH between 1 and 10, that requires 30 parts or less of water to dissolve one part of said film forming excipient, (ii) has a solubility in ethanol that requires 30 parts or less of ethanol to dissolve one part of said film forming excipient; and (iii) can prevent crystallization of said lidocaine and maintain said lidocaine in an amorphous state during solvent evaporation when said composition is applied to skin;
wherein said composition is a sprayable liquid solution; and wherein said composition contains less than 10% w/w water.

2. The composition of claim 1, wherein said composition further comprises one or more penetration enhancers.

3. The composition of claim 1, wherein the washability enhancer is selected from the group consisting of Polyethylene Glycol 300, Polyethylene Glycol 400, Polyethylene Glycol 600, and mixtures thereof.

4. The composition of claim 1, wherein said composition further comprises one or more viscosity increasing agents.

5. The composition of claim 1, wherein said composition comprises a unit dose of a volume of about 200 μl or less.

6. The composition of claim 1, wherein said composition is in a sealed container fitted with a spray pump.

7. A method of providing local anesthesia comprising spraying onto a subject's skin a volume of about 200 μL or less of the composition of claim 1.

8. The method of claim 7, wherein the volume of spray administered in a single actuation is about 90 μL.

9. The method of claim 8, wherein said volume of spray comprises about 40 mg lidocaine.

10. The method of claim 8, wherein said volume of spray comprises about 20 mg lidocaine.

11. The method of claim 7, wherein said composition leaves a film after the solvent evaporates.

12. A method of providing local anesthesia to a subject having post-herpetic neuralgia, comprising spraying the composition of claim 1 on an area of the subject's skin.

13. A method of providing local anesthesia to a subject having allodynia, comprising spraying the composition of claim 1 on an area of the subject's skin.

14. A method of providing anesthesia to an injection area of the skin prior to an injection, comprising spraying the composition of claim 1 on an area of the subject's skin.

15. A composition comprising:
a. at least 40% w/w lidocaine;
b. about 30% w/w to about 70% w/w of an aliphatic solvent selected from the group consisting of Acetone, Ethanol, Isopropyl Alcohol, and a mixture thereof;
c. about 1% w/w to about 5% w/w of a washability enhancer selected from the group consisting of Dibutyl Sebacate, Triethyl Citrate, Triacetin, Glycerol, Polyethylene Glycol 300, Polyethylene Glycol 400, Polyethylene Glycol 600, Propylene Glycol and a mixture thereof; and
d. about 0.1% w/w to about 10% w/w a film forming excipient, selected from the group consisting of Hydroxypropyl Cellulose, Ethyl Cellulose, Hydroxypropyl Methyl Cellulose, Amino Methacrylate Copolymer, Methacrylic Acid and Methyl Methacrylate Copolymer 1:1, Methacrylic Acid and Methyl Methacrylate Copolymer 1:2, Poly(butylmethacrylate-co-(2-dimethylaminoethyl)methacrylate-co-methyl methacrylate 1:2:1, a Methyl Vinyl Ether-Maleic Anhydride Copolymer, a Butyl Ester of Methyl Vinyl Ether/Maleic Anhydride Copolymer; a Polyvinyl Acetate-Povidone Polymer, and a mixture thereof;
wherein said composition is a sprayable liquid solution; and wherein said composition comprises less than 10% w/w water.

16. The composition of claim 15, wherein said composition further comprises about 0.1% w/w to about 10% w/w of a penetration enhancer selected from the group consisting of Azone, Glycerol Monooleate, Isopropyl Myristate, Octisalate, Oleic Acid, Diethylene Glycol Monoethyl Ether, and a mixture thereof.

17. The composition of claim 15, wherein the washability enhancer is selected from the group consisting of Polyethylene Glycol 300, Polyethylene Glycol 400, Polyethylene Glycol 600, and mixtures thereof.

18. The composition of claim 15, wherein said composition is capable of leaving a film on skin onto which the composition is sprayed after the solvent evaporates.

19. The composition of claim 15, wherein said composition comprises a unit dose of a volume of about 200 μl or less.

20. The composition of claim 15, wherein said composition is in a sealed container fitted with a spray pump.

21. A method of providing local anesthesia comprising spraying onto a subject's skin a volume of about 200 μL or less of the composition of claim 15.

22. The method of claim 21, wherein the volume of spray administered in a single actuation is about 90 μL.

23. The method of claim 22, wherein said volume of spray comprises about 40 mg lidocaine.

24. The method of claim 22, wherein said volume of spray comprises about 20 mg lidocaine.

25. The method of claim 21, wherein said composition leaves a film after the solvent evaporates.

26. A method of providing local anesthesia to a subject having post-herpetic neuralgia, comprising spraying the composition of claim 15 on an area of the subject's skin.

27. A method of providing local anesthesia to a subject having allodynia, comprising spraying the composition of claim 15 on an area of the subject's skin.

28. A method of providing anesthesia to an injection area of the skin prior to an injection, comprising spraying the composition of claim 15 on an area of the subject's skin.

29. A composition comprising:
a. at least 40% w/w lidocaine;
b. about 30% w/w to about 70% w/w Ethanol;
c. about 1% w/w to about 5% w/w Polyethylene Glycol 400; and
d. about 0.1% w/w to about 10% w/w a film forming excipient, selected from the group consisting of Hydroxypropyl Cellulose, Ethyl Cellulose, Hydroxypropyl Methyl Cellulose, Amino Methacrylate Copolymer, and a mixture thereof;
wherein said composition is a sprayable liquid solution; and wherein said composition comprises less than 10% w/w water.

30. The composition of claim 29, wherein said composition further comprises about 0.1% w/w to about 10% w/w of a penetration enhancer selected from the group consisting of Azone, Glycerol Monooleate, Isopropyl Myristate, Octisalate, Oleic Acid, Diethylene Glycol Monoethyl Ether, and a mixture thereof.

31. The composition of claim 29, wherein said composition is capable of leaving a film on skin onto which the composition is sprayed after the solvent evaporates.

32. The composition of claim 29, wherein said composition comprises a unit dose of a volume of about 200 μl or less.

33. The composition of claim 29, wherein said composition is in a sealed container fitted with a spray pump.

34. A method of providing local anesthesia comprising spraying onto a subject's skin a volume of about 200 μL or less of the composition of claim 29.

35. The method of claim 34, wherein the volume of spray administered in a single actuation is about 90 μL.

36. The method of claim 35, wherein said volume of spray comprises about 40 mg lidocaine.

37. The method of claim 35, wherein said volume of spray comprises about 20 mg lidocaine.

38. The method of claim 34, wherein said composition leaves a film after the solvent evaporates.

39. A method of providing local anesthesia to a subject having post-herpetic neuralgia, comprising spraying the composition of claim 29 on an area of the subject's skin.

40. A method of providing local anesthesia to a subject having allodynia, comprising spraying the composition of claim 29 on an area of the subject's skin.

41. A method of providing anesthesia to an injection area of the skin prior to an injection, comprising spraying the composition of claim 29 on an area of the subject's skin.

42. A composition comprising
a. about 2% w/w to about 50% w/w w/w lidocaine;
b. an aliphatic solvent;
c. about 0.1% w/w to about 10% w/w of a penetration enhancer;
d. about 0.1% w/w to about 5% w/w of a plasticizer;
e. a viscosity increasing agent; and
f. about 0.1% w/w to about 2% w/w of a flavoring agent;
wherein said composition is a sprayable liquid, and wherein said composition contains less than 10% w/w water.

43. The composition of claim 42, wherein the sprayable liquid is a gel or is capable of forming a gel upon contact with mucosal tissue.

44. The composition of claim 42, wherein said composition comprises a unit dose of a volume of about 200 μl or less.

45. A method of providing local anesthesia to a subject comprising spraying onto an area of gingiva or oral mucosa of the subject a volume of about 200 μL or less of the composition of claim 42.

46. The method of claim 45, wherein the volume of spray administered in a single actuation is about 50 μL.

47. The method of claim 46, wherein said volume of spray comprises about 2 mg to about 40 mg lidocaine.

48. The method of claim 46, wherein said volume of spray comprises about 20 mg lidocaine.

49. A composition comprising
a. at least 40% w/w lidocaine;
b. an aliphatic solvent;
c. about 0.1% w/w to about 10% w/w of a penetration enhancer;
d. about 0.1% w/w to about 5% w/w of a plasticizer; and
e. a viscosity increasing agent;
wherein said composition is a sprayable liquid solution; and wherein said composition contains less than 10% w/w water.

50. The composition of claim 49, wherein said penetration enhancer is selected from the group consisting of Azone, Glycerol Monooleate, Isopropyl Myristate, Octisalate, Oleic Acid, Diethylene Glycol Monoethyl Ether, and a mixture thereof.

51. The composition of claim 49, wherein the plasticizer is selected from the group consisting of Polyethylene Glycol 300, Polyethylene Glycol 400, Polyethylene Glycol 600, and a mixture thereof.

52. The composition of claim 49, wherein said composition comprises a unit dose of a volume of about 200 μl or less.

53. The composition of claim 49, wherein said composition is in a sealed container fitted with a spray pump.

54. A method of providing local anesthesia to a subject comprising spraying onto an area of pharynx mucosal lining of the subject a volume of about 200 μL or less of the composition of claim 49.

55. The method of claim 54, wherein said method is performed to prevent pain during an intubation or endoscopic procedure.

56. The method of claim 54, wherein the volume of spray administered in a single actuation is about 90 μL.

57. The method of claim 56, wherein said volume of spray comprises about 40 mg lidocaine.

58. The method of claim 56, wherein said volume of spray comprises about 20 mg lidocaine.

59. A composition according to any of claims 1 to 7 for use as a medicament.

60. A composition according to any of claims 1 to 7 for use in providing local anesthesia.

61. A composition according to any of claims 1 to 7 for use in providing local anesthesia to a subject having post-herpetic neuralgia.

62. A composition according to any of claims 1 to 7 for use in providing local anesthesia to a subject having allodynia.

63. A composition according to any of claims 1 to 7 for use in providing anesthesia to an injection area of the skin prior to an injection.

64. A composition according to any of claims 15 to 20 for use as a medicament.

65. A composition according to any of claims 15 to 20 for use in providing local anesthesia.

66. A composition according to any of claims 15 to 20 for use in providing local anesthesia to a subject having post-herpetic neuralgia.

67. A composition according to any of claims 15 to 20 for use in providing local anesthesia to a subject having allodynia.

68. A composition according to any of claims 15 to 20 for use in providing anesthesia to an injection area of the skin prior to an injection.

69. A composition according to any of claims 29 to 33 for use as a medicament.

70. A composition according to any of claims 29 to 33 for use in providing local anesthesia.

71. A composition according to any of claims 29 to 33 for use in providing local anesthesia to a subject having post-herpetic neuralgia.

72. A composition according to any of claims 29 to 33 for use in providing local anesthesia to a subject having allodynia.

73. A composition according to any of claims 29 to 33 for use in providing anesthesia to an injection area of the skin prior to an injection.

74. A composition according to any of claims 42 to 44 for use as a medicament.

75. A composition according to any of claims 42 to 44 for use in providing local anesthesia to an area of gingiva or oral mucosa.

76. A composition according to any of claims 49 to 53 for use as a medicament.

77. A composition according to any of claims 49 to 53 for use in providing local anesthesia to an area of pharynx mucosal lining.
